(19) United States
(12) Patent Application Publication
(10) Pub. No.: US 2020/0247732 A1
(43) Pub. Date: Aug. 6, 2020

(54) HIGHLY EFFICIENT ACID CATALYST FOR HYDROCARBON CONVERSION

(71) Applicant: Viridis Chemicals Private Limited, Mumbai (IN)

(72) Inventors: Chaitanya SAMPARA, Mumbai (IN); Vivek KHACHANE, Mumbai (IN)

(21) Appl. No.: 16/646,619

(22) PCT Filed: Sep. 12, 2017
(86) PCT No.: PCT/IN2017/050394

§ 371 Date: Mar. 12, 2020

(51) Int. Cl. C07C 5/22; C07C 2/86; B01J 27/055

(52) U.S. Cl. CPC C07C 5/2213; C07C 2/861; C07C 2521/06; C07C 2527/053; B01J 27/055

(57) ABSTRACT

A mixed metal oxide solid acid catalyst composition is disclosed which provides substantially improved conversion for hydrocarbon transformation reactions namely, alkylation and isomerization. The catalyst composition includes a sulfate ion, Platinum group metal and a mixed metal oxide support material bearing molecular formula:

x₁ZrO₂.x₂Al₂O₃.x₃Yb₂O₃.x₄CuO

wherein the molar coefficients for individual metal oxides are as follows:
x1=55 to 75×10⁻²; x2=12 to 25×10⁻²; x3=1 to 6×10⁻² and x4=0.1 to 5×10⁻²;

The concentration of the sulfate ion on the aforementioned catalyst support is between 5 to 17 wt % and that of Platinum group metal is 0.05 to 2.0 wt %.

### FIELD OF INVENTION

The present disclosure relates to a highly efficient sulfated mixed metal oxide based solid acid catalyst composition and a process for the preparation thereof to improve the reactant conversion for hydrocarbon processes, more specifically aimed towards hydrocarbon alkylation and isomerization.

### BACKGROUND OF INVENTION

Solid acid catalysts find their rampant use in the acid catalyzed organic reactions due to their superiority over the conventional acid catalysts such as less corrosive, less toxic, environmentally friendly, easy to handle, recoverable and reusable. Though, the plenty of solid acid catalysts such as zeolites, heteropolyacids, ion exchange resins, clays and the like are reported for various catalytic conversion processes, zirconia based solid acid catalysts have drawn much attention in the recent years due to their extraordinary properties.

The most common method of producing sulfated zirconium oxide based catalysts involves precipitation of zirconium salt using common bases such as but not limited to NH₄OH, NaOH, KOH and the like to make zirconium hydroxide. The zirconium hydroxide is then impregnated with sulfur containing precursors such as H₂S, (NH₄)₂SO₄, H₂SO₄, Na₂S₂O₈, mercaptans or SO₂ which are capable of forming sulfate ions and the like to obtain a sulfated zirconium hydroxide. The sulfated zirconium hydroxide is then calcined at high temperature and further impregnated with noble metals such as platinum, palladium, Iridium and the like which constitutes the hydrogenating component of the catalyst. The catalysts produced by this classic method lack high surface area and active sites which are required for consistence catalytic performance in the reaction system. In addition, the active sites often degrade over extended operating times which are common for the acid catalyzed reaction systems.

Further, the use of several dopant systems is reported to increase the stability of these catalyst compositions, for example, Russian patent document 2191627 discloses a method for the synthesis of sulfated zirconia catalyst loaded with noble metals such as platinum, palladium, ruthenium, osmium, iridium and the like on an zirconia support containing up to 20% of active components of silicon, titanium oxide, magnesium and alumina. The catalyst is further loaded with Nickel, Titanium, Germanium, Manganese, Cobalt, Bismuth, Iron, Vanadium, Cobalt, Zirconium and mixtures thereof. The catalyst as disclosed in the aforementioned patent is used for the isomerization reaction; however, the catalyst demonstrates very poor stability. The concentration of 2,2,-dimethylbutane (2,2-DMB), a key performance product decreases after 200 hours of operation from 28% to 14%.

European patent document 1002579 discloses a layered catalyst having zirconium core component followed by Mn, Fe or Ni shell and a top layer consisting of noble metals. The catalyst was used for the isomerization reaction. However, the catalyst shows degradation in overall performance after 200 hours of operation. The concentration of 2,2-DMB, a key performance product decreases from 28% to 20%.

Another Russian patent document 2171713 discloses a process for the preparation of a catalyst with 0.2 to 1% platinum or Palladium, 0.05 to 2.5% chlorine and 0.5 to 10% sulfate which are deposited on a mixture of aluminum and zirconium oxide. The major disadvantage of this catalyst is low stability for isomerization reaction. This catalyst also shows reduction in the concentration of 2,2-DMB from 34% to 25% after 200 hours of operation.

United States Patent document 7015175 discloses a method for the synthesis of sulfated zirconium catalyst doped with high concentration (of at least 3%) of Lanthanide series elements. The performance of the optimized catalyst is shown in terms of conversion of n-pentane and cyclohexane and not with the classically described performance parameter 2,2-DMB. In addition, no description is provided on the stability of the catalyst over prolonged usage.

Therefore, there is felt a need to provide a highly efficient sulfated zirconium oxide based solid acid catalyst composition which retains high catalytic activity and sustain high surface area over wide and extended operating conditions.

### SUMMARY AND OBJECTS OF INVENTION

A purpose of the present invention is to provide an improved solid acid catalyst for high reactant conversion for hydrocarbon processes, specifically for alkylation and isomerization. The catalyst is based on the sulfated mixed metal oxide support composition that has superior performance and stability in comparison to existing catalysts reported in the open literature.

A broad embodiment of the present invention is directed to the catalyst support composition bearing molecular formula:

x₁ZrO₂.x₂Al₂O₃.x₃Yb₂O₃.x₄CuO

wherein the molar coefficients for individual metal oxides are as follows:
x1=55 to 75×10⁻²; x2=12 to 25×10⁻²; x3=1 to 6×10⁻² and x4=0.1 to 5×10⁻²;

The support bearing the aforementioned composition is sulfated with a sulfate ion (SO₄²⁻) having a weight percentage ranging between 5 to 17 wt %. Further, a Platinum group metal is doped on the said catalyst with a weight percentage ranging between 0.05 to 2%.

Some of the summary or objects of the present disclosure are described herein below:

It is an object of the present disclosure to ameliorate one or more problems of the prior art or to at least provide a useful alternative.

Another object of the present disclosure is to provide a highly efficient and stable sulfated mixed metal oxide based solid catalyst composition.

Still another object of the present disclosure is to provide a sulfated mixed metal oxide based catalyst composition wherein the catalyst demonstrates excellent activity and retains its activity, acidity and high surface area even over wide and extended operating conditions.

Yet another object of the present disclosure is to provide a process for the preparation of a sulfated mixed metal oxide based catalyst.

Further object of the present disclosure is to provide hydrocarbon conversion processes carried out by using a sulfated mixed metal oxide based solid acid catalyst composition.

Other objects and advantages of the present disclosure will be more apparent from the following description which is not intended to limit the scope of the present disclosure.

### DETAILED DESCRIPTION OF THE INVENTION

In a first aspect, the present disclosure provides a sulfated mixed metal oxide based solid acid catalyst composition used for the catalytic conversion of hydrocarbons.

In accordance with one of the exemplary embodiments of the present disclosure, the sulfated zirconium oxide based solid catalyst composition comprises:
i. Synthesis of high surface area Zirconium hydroxide support in the presence of sulfate, Ytterbium and Copper elements at temperatures less than 20° C. with controlled addition of base.
ii. a sulfated solid support comprising Sulfate ion (SO₄²⁻) in an amount ranging between 5 to 17 wt-%, wherein said support comprises hydroxides of zirconium, aluminum, ytterbium and copper. In the above definition MOx denotes the mixed metal oxide
iii. at least one lanthanide series element wherein the Lanthanide element:Zr concentration ranges between 0.01 to 2.35 mole %; and
iv. The final catalyst support (excluding the sulfate ion) bearing molecular formula

x₁ZrO₂.x₂Al₂O₃.x₃Yb₂O₃.x₄CuO
wherein the molar coefficients for individual metal oxides are as follows:
x1=55 to 75×10⁻²; x2=12 to 25×10⁻²; x3=1 to 6×10⁻² and x4=0.1 to 5×10⁻²;
v. Platinum group metal including but not limited to Platinum, Pallladium, Iridium, Rhenium, Rhodium and Ruthenium is then spray impregnated on the sulfated support in a weight percentage of 0.05 to 2 wt %.
vi. The catalyst thus prepared is then calcined at a temperature 300° to 800° C. and formed into the desired shape as desired by the specific hydrocarbon conversion process (alkylation or isomerization).

The sulfated mixed metal oxide based catalyst composition in accordance with the present disclosure comprises a solid support comprising oxides of zirconium, aluminum, ytterbium and copper. The solid support comprising oxides of the aforementioned metals (i.e. zirconium, aluminum, ytterbium and copper) is sulfated with sulfur and further loaded with platinum group metals.

As described above, the metal loaded in the sulfated zirconium oxide based catalyst composition of the present disclosure is present in an amount varying between 0.05 to 2.0 wt %, based on the total molar mass of the final catalyst. The amount of each of above described metals loaded in the catalyst composition of the present disclosure is not same and can be varied according to the type of the metal loaded.

In accordance with another exemplary embodiment of the present disclosure, the sulfated zirconium oxide based catalyst composition further comprises at least one noble metal selected from the group consisting of Ru, Re, Rh, Ir, Pd and Pt.

In another aspect, the present disclosure provides a process for the preparation of a sulfated mixed metal oxide based catalyst composition.

The process for the preparation of the sulfated mixed metal oxide based solid catalyst composition in accordance with the present disclosure comprises the steps of reacting a zirconium and an aluminum precursor in the presence of a lanthanide series element, specifically ytterbium, copper and a sulfate species to obtain a solid support comprising hydroxides of zirconium and aluminum, depositing sulfur and a metal on the solid support to obtain sulfated zirconium oxide based catalyst composition of the present disclosure.

In accordance with one of the exemplary embodiments of the present disclosure, the process for the preparation of a sulfated zirconium oxide based catalyst composition comprising the following steps:
i. reacting at least one of aluminum precursor and zirconium precursor in the presence of at least one ytterbium and copper, and at least one sulfate species to obtain a solution;
ii. introducing a base solution to the salt solution at a pre-determined temperature to precipitate a product comprising hydroxides of either zirconium or aluminum or both;
iii. Drying the precipitate after removal of unwanted ions by washing.
iv. Additionally adjusting the sulfate content in the catalyst using sulfating agents to obtain a sulfated product having sulfur content in the range of 5 to 17 wt %, based on the total mass of the final product; and
v. calcining the metal loaded sulfated product to obtain a sulfated zirconium oxide based catalyst composition at a temperature of 300° to 800° C.
vi. Optionally depositing noble metals from the group of Ru, Rh, Pt, Pd and Ir on the catalyst depending on the final application of the product.
vii. The final catalyst can also be formed into suitable physical forms such as pellets or extrudes to increase the hydrocarbon reaction efficacy under test.

The aluminum precursor suitable for the purpose of the present disclosure includes, but is not limited to, chlorides, nitrates, pseudo bohemite and/or sulfates of aluminum. Similarly, the suitable examples of zirconium precursor in accordance with present disclosure include, but are not limited to, chlorides, oxychlorides, nitrates and sulfates of zirconium.

In accordance with one of the exemplary embodiments of the present disclosure, the molar proportion of aluminum to zirconium metal precursor (Al:Zr) varies between 0.00 and 5%; molar proportion of the lanthanide series element varies between 0.001 and 3%; molar proportion of the copper varies between 0.001 to 3% molar and the molar proportion of sulfate species to zirconium metal precursor varies between 0.03 and 4.3%.

The sulfate species used for the purpose of the present disclosure is the conventionally used sulfate species. However, the suitable examples of such sulfate species for the purpose of the present disclosure include, but are not limited to H₂S, mercaptans or SO₂ which can provide sulfate ions, H₂SO₄ (NH4)₂SO₄ and Na₂S₂O₈.

The method step of depositing the metal on the sulfated product in accordance with the process of the present disclosure is carried out by treating the sulfated product with at least one metal precursor. The metal precursor suitable for the purpose of the present disclosure includes, but is not limited to, nitrates, sulfates, chlorides and acetates. The metal may be deposited on the sulfated product by employing methods known in the prior-art.

The metal loaded sulfated product in accordance with the present disclosure is calcined at a temperature varying between 300 and 800° C. for a pre-determined period of time to obtain a final sulfated zirconium oxide based catalyst composition. The calcination of the sulfated product is performed in different stages.

In accordance with one of the embodiments of the present disclosure, the metal loaded sulfated product is calcined at two different stages. The first stage calcination is carried out at a temperature varying between 300 and 550° C. and at a rate varying between 1 and 5° C./min whereas the second stage calcination is carried out at a temperature varying between 550 and 800° C. and at rate varying between 1 and 10° C./min.

The calcination at different stages provides a uniform crystal structure. The sulfated mixed metal oxide based catalyst composition in accordance with the present disclosure comprises teteragonal structure which is required for high catalytic activity. The surface area of the sulfated zirconium oxide based catalyst composition in accordance with the one of the exemplary embodiments of the present disclosure varies between 100 and 150 m²/g.

The process for the preparation of the sulfated metal oxide based catalyst composition in accordance with the present disclosure further comprises a method step of impregnating at least noble metal on the sulfated zirconium oxide based catalyst composition. The noble metal in accordance with the present disclosure includes, but is not limited to Ru, Rh, Pd, Pt and Ir. The noble metal may be impregnated by using methods commonly known in the prior-art. The impregnation of the noble metal is carried out in such a way so that the final particle size of the sulfated zirconium oxide based catalyst is less than 4 nm, as measured by TEM.

The sulfated mixed metal oxide based catalyst composition of the present disclosure is used in various hydrocarbon conversion processes, specifically for alkylation and isomerization.

In still another aspect, the present disclosure provides hydrocarbon conversion processes that include, but are not limited to, isomerization, dimerization, alkylation and acylation by using a sulfated zirconium oxide based catalyst composition of the present disclosure.

In accordance with one of the exemplary embodiments of the present disclosure, the sulfated zirconium oxide based catalyst composition of the present disclosure is used for the alkylation and isomerization reactions, the results of which are provided below in Table-1 and Table-2.

Table-1: Alkylation of Toluene with Benzyl Chloride:

The catalyst was calcined at a temperature between 400°-600° C. for 4 hours and reduced in H₂ atmosphere for 2 hours at a temperature between 150°-350° C. before following reaction was conducted. Reactants are charged in the reaction chamber fitted with a condenser in the amounts described in table 1. The reactants are brought to reflux and the reaction is conducted for 1.5 hours. The reactants are then cooled the products are analyzed using a Gas Chromatograph with an OV-101 column.

TABLE 2
Isomerization:

| | | |
|---|---|---|
| Toluene | 37.5 | gms |
| Benzyl chloride | 9.5 | gms |
| Catalyst used | 0.09 | gms |

| | |
|---|---|
| Conversion of Benzyl chloride | 100% (after 1.5 hour of operation) |
| Selectivity to di-phenyl benzene | 99.7% |

The catalyst was calcined at a temperature between 400°-600° C. for 4 hours and reduced in H₂ atmosphere for 2 hours at a temperature between 150°-350° C. before following reaction was conducted. The catalyst synthesized as described previously was loaded in a fixed bed reactor at the desired temperature and pressure. The reactant feed was vaporized before contacting with the catalyst and the products were cooled using a gas liquid separator. The products and reactants were analyzed using a gas chromatograph with OV-101 column.

| | Concentration and operating conditions | |
|---|---|---|
| n-pentane | 35% | |
| n-hexane | 53% | |
| Cyclohexane | 10% | |
| n-heptane | 2% | |
| H₂/HC | 1-4 | |
| Pressure | 20-38 | MPa |
| Temperature | 150-180° | C. |
| 2,2,-dimethylbutane | 35.4% | |
| (2,2-DMB)/C6 (1 hr) | | |
| 2,2-DMB/C6 (500 hrs) | 35.5% | |
| WHSV | 2 | h⁻¹ |

It is evident from the data of Table-1 and Table-2 that the sulfated zirconium oxide based catalyst composition of the present disclosure shows high activity and retains the same over wide and extended operating conditions.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### TECHNICAL ADVANCEMENT

The present disclosure relates to a sulfated mixed metal oxide based catalyst composition and a process for the preparation thereof, has several technical advancements that include, but are not limited to, the realization of high catalytic activity and high stability over wide and extended operating conditions for hydrocarbon conversion processes, specifically alkylation and isomerization.

1. A method of converting hydrocarbons into high value products by isomerization and alkylation by contacting the reactants with a catalyst containing Group 3A, 4A, 11 and 8 elements along with Platinum group metals and sulfated ion, at optimized temperature and pressure conditions as desired for the hydrocarbon process, wherein the catalyst contains the following composition of metal oxides:
x₁ZrO₂.x₂Al₂O₃.x₃Yb₂O₃.x₄CuO
wherein the mole-coefficients for the individual oxides are as follows:
x1=55 to 75×10⁻²; x2=12 to 25×10⁻²; x3=1 to 6×10⁻² and x4=0.1 to 5×10⁻²;
and the mass ratio of sulfated ion is between 5 to 17% and that of Platinum group metals is between 0.05 to 2%.
a. a sulfated metal oxide (MOx) comprising sulfur in an amount ranging between 0.01 to 7 mole %, said metal oxide is at least one metal oxide selected from group consisting of zirconium oxide, aluminum oxide, ytterbium oxide and copper oxide.
b. at least one lanthanide series element, specifically ytterbium, in a molar concentration ratio ((Lanthanide element:Zr) ranging between 0.01 to 2.35 mole %;
c. at least one additional metal selected from the group consisting of Cu, Bi, Ti, Fe, Mn, Co and Ni in an amount ranging between 0.0 to 1.0 mole %, wherein each of said molar proportions being with respect to the total molar mass of final catalyst.
d. at least one additional metal selected from the Platinum group metals including Ru, Re, Rh, Ir, Pd and/or Pt in an amount ranging between 0.05 to 2 wt % based on the final catalyst composition.

2. A method of hydrocarbon conversion according to claim 1, characterized by sulfur ion is present on the catalyst in the form of SO₄²⁻ or SO₃²⁻ ions.

3. A method of hydrocarbon conversion according to claim 1, wherein the overall weight percentage of the catalyst composition is as follows:

| | |
|---|---|
| [symbol] r SO₃²⁻ ion | [symbol] % |
| 8A metal | [symbol] 2% |
| [symbol] oxide support | [symbol] e to 100% |

[symbol]

indicates data missing or illegible when filed

4. A method of hydrocarbon conversion, specifically by isomerization according to claim 1, characterized in that the hydrogenating component of the catalyst use at least 2 metals from Group 8A of the periodic table comprising of Platinum, Palladium, Iridium, Rhodium and/or Ruthenium.

5. A method of hydrocarbon conversion according to the catalyst described in claim 1, wherein the isomerization process is conducted at a temperature range of 120 to 220° C., a pressure of 1.5 to 4.0 MPa and a hydrogen to hydrocarbon ratio of 1-10:1

6. A method of hydrocarbon conversion according the catalyst described in claim 1, wherein alkylation process is conducted at a temperature range of 90° to 240° C. depending on the hydrocarbon reactant.
